(19) United States
(12) Patent Application Publication
(10) Pub. No.: US 2011/0160569 A1
(43) Pub. Date: Jun. 30, 2011

(54) SYSTEM AND METHOD FOR REAL-TIME SURFACE AND VOLUME MAPPING OF ANATOMICAL STRUCTURES

(76) Inventors: Amit Cohen, Binyamina (IL); Itay Kariv, Haifa (IL)

(21) Appl. No.: 12/651,031

(22) Filed: Dec. 31, 2009

(51) Int. Cl. A61B 6/00

(52) U.S. Cl. 600/424

(57) ABSTRACT

A method and system for mapping a volume of an anatomical structure includes a processor for computing a contour of a medical device as a function of positional and/or a shape constraints, and to translate the contour into known and virtual 3D positions. The processor is configured to determine a spatial volume based on a virtual position, and to render a 3D representation of the spatial volume. A method and system of mapping a surface of an anatomical structure includes a processor configured to obtain an image of the structure. The processor is further configured to receive a signal indicative of a medical device contacting the surface of the anatomical structure, and to determine a position of the device upon when contact has been made. The processor is configured to superimpose marks on the image indicative of a contact points between the device and the structure.

### BACKGROUND OF THE INVENTION

a. Field of the Invention

The present disclosure relates to a system and method for real-time surface and volume mapping of an anatomical structure.

b. Background Art

In a cannulation procedure, a physician cannulates into a vessel of an anatomical structure, such as, for example, a vessel of the heart. In such procedures, a physician or clinician inserts a medical device or tool, such as, for example, a catheter, into an insertion region of a patient, which, in an exemplary embodiment, may comprise the Superior Vena Cava (SVC) of the heart. Once the medical device or tool is inserted, the physician or clinician uses the device or tool to probe around a surface of the anatomical structure searching for the ostium of the vessel the physician is attempting to cannulate through.

One drawback of known cannulation methodologies is that the procedures such as this can be rather lengthy procedures that employ a “trial and error” method for searching for the ostium (i.e., the physician “pokes” or probes around the surface until the ostium is found). Without information regarding where the physician previously poked or probed, the same area may be poked or probed several times, thereby lengthening the time of the procedure and increasing the amount of radiation exposure (i.e., x-ray) needed, thus resulting in exposing the anatomical structure or region of interest to redundant irritation.

Lengthy procedure times is also a drawback of known or conventional volume mapping systems/methodologies. Various systems and methods for mapping volumes of anatomic structures are generally known in the art. In certain medical procedures, such as, for example, lead place or tissue ablations, there is a need for detailed mapping of an anatomical structure or region of interest. Such mapping provides the ability to navigate medical devices or tools, such as catheters, to specific targets. Known systems include both magnetic field-based systems, as well as non-magnetic field-based systems. These systems/methodologies may include moving a medical device or tool within a region of interest of a patient's anatomy and collecting position and orientation information of one or more positioning sensors associated with the medical device. The system continuously analyzes the location and orientation information, and then using the information, generally provides a substantially real-time map or model of an anatomical structure disposed within the region of interest. This map or model may then be displayed on, for example, a computer monitor for a user to use in connection with navigation, for example, within the anatomical structure or region of interest.

As with the known cannulation procedures described above, one disadvantage with these known systems is that in order to reproduce the actual anatomy with all of its complexities, as well as to extract relevant characteristics of the anatomical structure to provide real-time parametric displays for evaluation and treatment, the mapping process is excessively long or time consuming. More particularly, these processes typically require the collection of up to hundreds of valid position and orientation data points. As a result, the mapping process is long and drawn out, thereby causing a procedure being performed in conjunction with the mapping procedure to be unnecessarily lengthened.

Accordingly, there is a need for a system that will minimize and/or eliminate one or more of the above-identified deficiencies.

### BRIEF SUMMARY OF THE INVENTION

The present invention is directed to a system and method for real-time surface and volume mapping of anatomical structures. In accordance with one aspect of the present teachings, a system for three-dimensionally mapping a volume within a region of interest (ROI) of body is provided. The system includes a processor. The processor is configured to compute a contour for a medical device disposed within the ROI as a function of at least one of a positional constraint and a shape constraint. The processor is further configured to translate the contour into a plurality of three-dimensional positions wherein the plurality of three-dimensional positions include both known and virtual positions. The processor is still further configured to determine a real-time spatial volume based on at least one virtual three-dimensional positions, and to render a real-time three-dimensional graphical representation of the spatial volume.

In accordance with another aspect of the present teachings, a method for three-dimensionally mapping a volume within a region of interest (ROI) of a body is provided. The method includes the step of tracking the position of an invasive medical device within the ROI in real-time. The tracking step comprises the substeps of computing a contour for the medical device as a function of at least one of a positional constraint and a shape constraint, and translating the computed contour into a plurality of three-dimensional position, wherein the three-dimensional positions include both known and virtual positions. The method further includes the step of determining a real-time spatial volume based on at least one virtual three-dimensional position, and the step of rendering a real-time three-dimensional graphical representation of the spatial volume.

In accordance with yet another aspect of the present teachings, a system for mapping a surface of an anatomical structure is provided. The system includes a processor configured to obtain an image of the anatomical structure. The processor is further configured to receive a signal indicative of a medical device contacting the surface of the anatomical structure. The processor is still further configured to determine a real-time position of the medical device responsive to the signal when the signal is indicative of the medical device contacting the anatomical structure. The processor is yet still further configured to superimpose on the image a mark corresponding to the position of the medical device to indicate that the anatomical structure was contacted by the medical device at the point on the anatomical structure where the mark is disposed.

In accordance with yet still another aspect of the present teachings, a method for mapping a surface of an anatomical structure in real-time is provided. The method includes the step of obtaining an image of the anatomical structure. The method further includes determining a real-time position of a medical device when the medical device contacts the anatomical structure. The method still further includes superimposing, on the image, a mark corresponding to the position of the medical device to indicate the anatomical structure was contacted at the point on the anatomical structure where the mark is disposed.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic and block diagram view of an exemplary embodiment of a system for mapping a volume of an anatomical structure in accordance with the present teachings.

FIG. 2 is a schematic and block diagram view of another exemplary embodiment of the system illustrated in FIG. 1.

FIG. 3a is a diagrammatic view of an exemplary medical device in accordance with the present teachings.

FIG. 3b is a diagrammatic view of a representation of a contour of the medical device illustrated in FIG. 3a.

FIGS. 4a and 4b are diagrammatic views of medical devices disposed within a region of interest.

FIG. 5 is a flow chart illustrating an exemplary method of mapping a volume of an anatomical structure in accordance with the present teachings.

FIG. 6 is a diagrammatic view of a portion of the system illustrated in FIG. 1 used in connection with time-dependent gating.

FIG. 7a is a diagrammatic and block diagram view of a system for mapping a surface of an anatomical structure in accordance with the present teachings.

FIG. 7b is a schematic and block diagram view of another exemplary embodiment of the system illustrated in FIG. 1.

FIG. 8 is a diagrammatic view of an image displayed on a display device having contact points, a corresponding surface map, and a representation of a medical device superimposed thereon.

FIG. 9 is a flow chart illustrating an exemplary embodiment of a method of mapping a surface of an anatomical structure in accordance with the present teachings.

FIG. 10 is a schematic and block diagram view of one exemplary embodiment of a medical positioning system (MPS) as shown in block form in FIGS. 1, 2, 7a, and 7b.

### DETAILED DESCRIPTION OF THE INVENTION

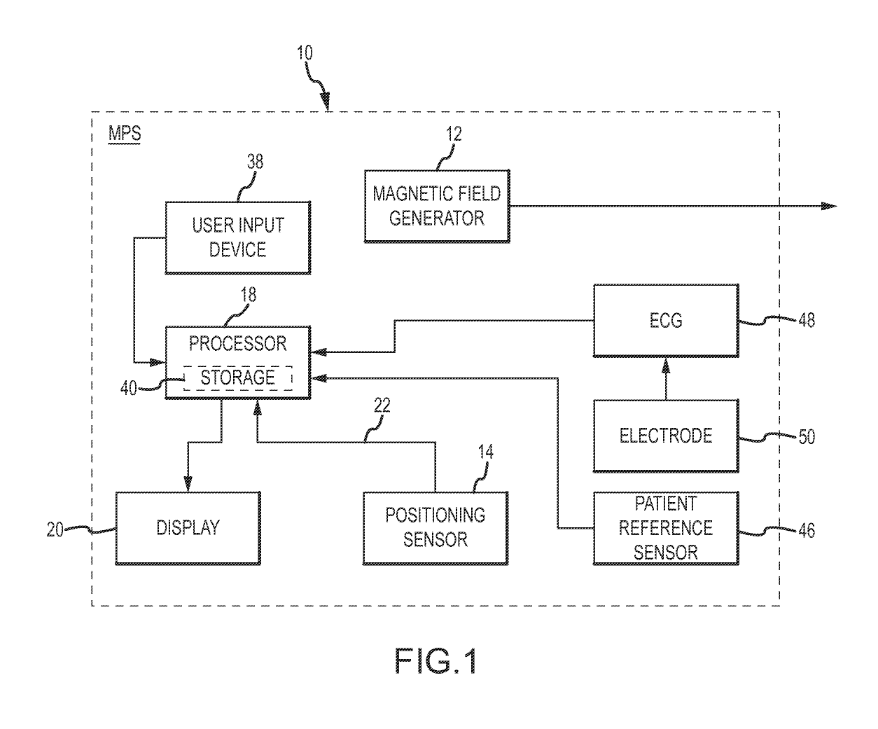

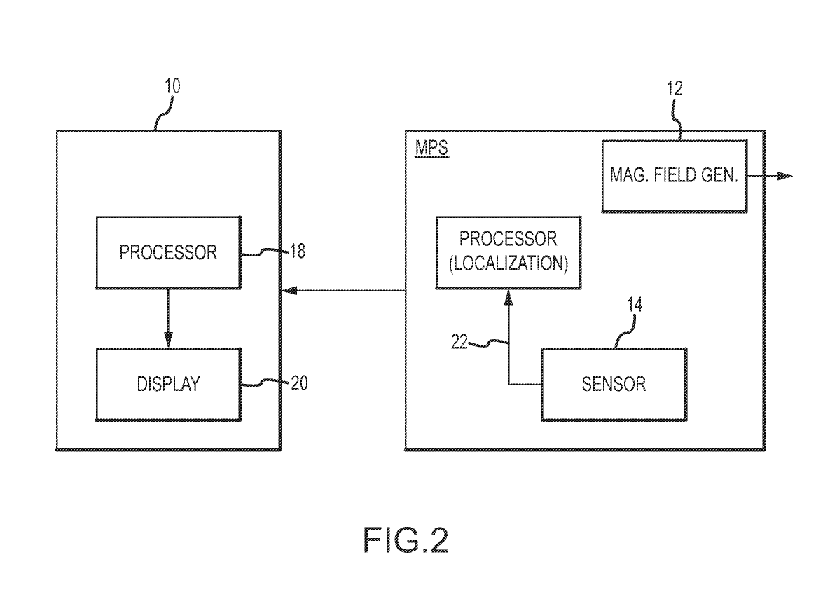

Referring now to the drawings wherein like reference numerals are used to identify identical components in the various views, FIG. 1 illustrates an exemplary embodiment of a system 10 for mapping a volume of an anatomical structure within a region of interest (ROI) of a patient's body. In an exemplary embodiment, the system 10 comprises a medical positioning system (MPS). It should be noted, however, that in other exemplary embodiments, such as that illustrated in FIG. 2, rather than comprising an MPS, the system 10 is separate and distinct from the MPS but configured for use in conjunction with an MPS.

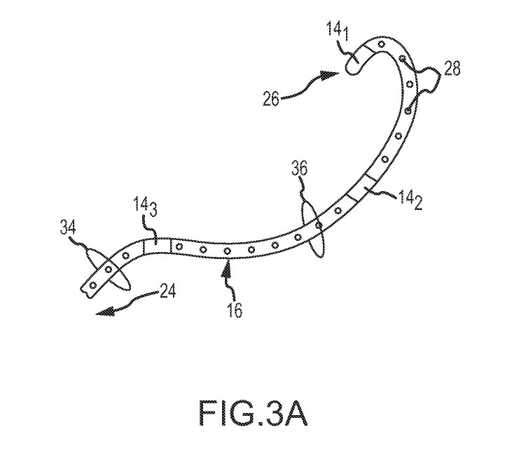

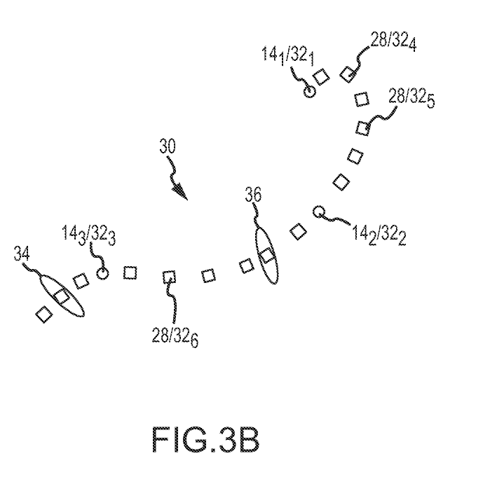

In either embodiment, the MPS is configured to serve as a localization system, and therefore, is configured to determine positioning (localization) data with respect to one or more MPS sensors and to output a respective location reading. The location readings may each include at least one or both of a position and an orientation (P&O) relative to a reference coordinate system, which may be the coordinate system of the MPS. In the embodiments illustrated in FIGS. 1 and 2, and as will be described in greater detail below, the MPS is a magnetic field-based MPS. In such an embodiment the P&O may be expressed as a position (i.e., a coordinate in three axes X, Y, and Z) and an orientation (i.e., an azimuth and elevation) of the sensor (i.e., a magnetic field sensor) in the magnetic field relative to a magnetic field generator(s)/transmitter(s). Other expressions of a P&O (e.g., other coordinate systems) are known in the art and fall within the spirit and scope of the present invention (e.g., see, for example, FIG. 3 and associated text of U.S. Pat. No. 7,343,195 entitled “Method and Apparatus for Real Time Quantitative Three-Dimensional Image Reconstruction of a Moving Organ and Intra-Body Navigation,” which is incorporated herein by reference in its entirety, viz. location [X, Y, Z] and orientation (angles α, β, χ)).

For ease of description purposes only, the system 10 will be hereinafter described as comprising, or being configured for use in conjunction with, a magnetic field-based MPS (as opposed to a non-magnetic field-based system). It should be noted, however, that while the description below is primarily focused on a magnetic field-based MPS, the present invention is not meant to be limited to such a type of MPS. Rather, one of ordinary skill in the art will appreciate that the present invention may also be implemented as an MPS other than a magnetic field-based MPS. For example, the MPS may comprise an electric field-based system. One example of an electric field-based system is the EnSite NavX™ System commercially available from St. Jude Medical, Inc. and as generally shown with reference to U.S. Pat. No. 7,263,397 entitled “Method and Apparatus for Catheter Navigation and Location and Mapping in the Heart,” which is incorporated herein by reference in its entirety. Accordingly, the present invention is not limited to any particular type of MPS, or any specific implementation of a particular type of MPS.

With continued reference to FIGS. 1 and 2, in an embodiment wherein the system 10 comprises a magnetic field-based MPS, the system 10 generally includes a magnetic field generator/transmitter assembly 12, one or more positioning sensors 14 associated with a medical device or tool 16 (collectively referred to hereinafter as “medical device 16” and best shown in FIG. 3, a processor 18, and a display device 20.

The magnetic field generator/transmitter assembly 12 is configured to generate one or more controlled AC magnetic fields in and around an area of interest of a patient's body in a predefined three-dimensional space. The characteristics of the generated magnetic field(s) are such that the field(s) are used, as briefly described above, to acquire positioning data (i.e., P&O) of one or more magnetic field positioning sensors, such as positioning sensors 14, and therefore, at least a portion of the medical device 16 with which the sensors 14 are associated. Among other things, the P&O of the positioning sensors 14 may be used to track the position of the sensors and the associated medical device 16.

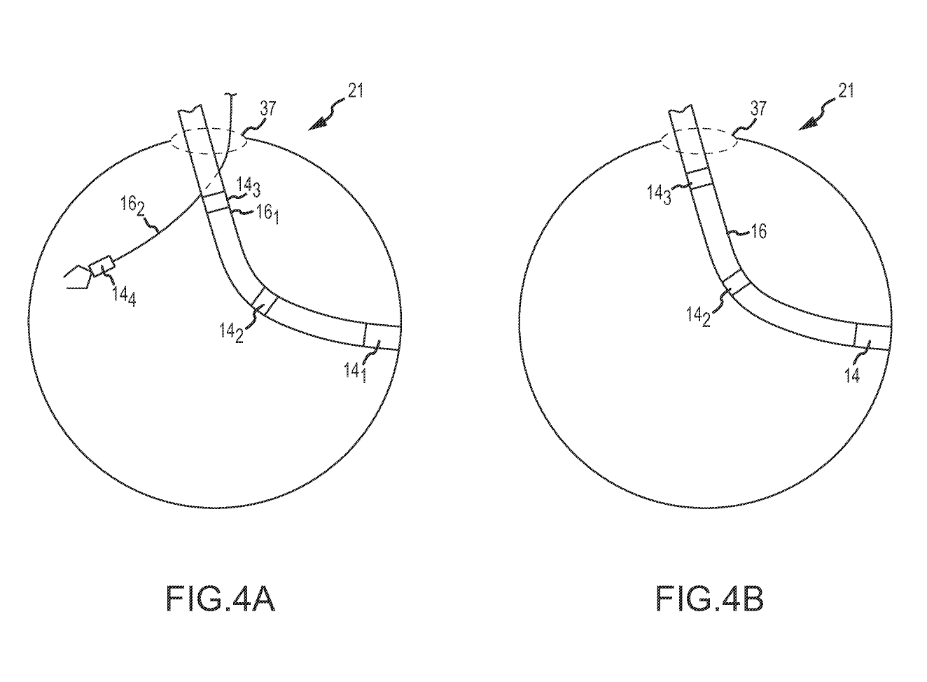

As briefly described above, and with reference to FIGS. 3a, 4a, and 4b, the system 10 may comprise one or more positioning sensors 14 (14₁, 14₂, . . . , 14_{N}) associated with the medical device 16. Additionally, as illustrated in FIG. 4a, the system 10 may comprise multiple medical devices (i.e., medical devices 16₁, 16₂, . . . , 16_{N}) each having one or more positioning sensors associated therewith. In an embodiment wherein the system 10 includes a plurality of positioning sensors 14, each sensor 14 would be tracked within the ROI (referred to hereinafter as ROI 21) independently, and therefore, its position would be tracked independently. In the interest of clarity and brevity, the following description will be directed to an embodiment such as that illustrated in FIG. 4b having a single medical device 16 with a plurality of positioning sensors 14 associated therewith. It should be noted, however, that embodiments of the system 10 having more than one medical device remain within the spirit and scope of the present invention.

As illustrated in FIGS. 3a and 4b, in an exemplary embodiment the medical device 16 includes a plurality of positioning sensors 14. For purposes to be described below, each sensor 14, and more particularly, the location on the medical device 16 corresponding thereto, represents a known point of the medical device 16. Each positioning sensor 14 may take the form of any number of magnetic field sensors known in the art. In an exemplary embodiment, sensors 14 comprise one or more magnetic field detection coil(s). In such an embodiment, voltage is induced on the coil(s) when the coil(s) are disposed within a changing magnetic field. For one example of a sensor, see U.S. Pat. No. 7,197,354 entitled “System for Determining the Position and Orientation of a Catheter,” which is incorporated herein by reference in its entirety. It should be noted that variations as to the number of coils, their geometries, spatial relationships, the existence or absence of cores, and the like are possible and all remain within the spirit and scope of the present invention.

Each positioning sensor 14 is configured to detect a magnetic field generated by the magnetic field generator/transmitter 12, and to generate or produce a signal 22 (hereinafter referred to as “positioning signal 22”) representative of the detected magnetic field. More particularly, each positioning sensor 14 is configured to detect one or more characteristics of the magnetic field(s) in which it is disposed and to generate a positioning signal 22 that is indicative of the detected characteristic(s). Each signal 22 is then processed by the MPS to obtain a respective P&O thereof.

Accordingly, as will be described in greater detail below, the positioning signal(s) 22 generated or produced by the positioning sensor(s) 14 are communicated to a processor of the MPS and are used by the processor to determine or calculate the P&O of each positioning sensor 14, and therefore, a portion of the medical device 16 corresponding to the location of the positioning sensor 14 on the medical device 16. In an exemplary embodiment, the processor 18 is configured to carry out this functionality. Accordingly, in such an embodiment, each positioning sensor 14 is electrically connected to the processor 18. Alternatively, in another exemplary embodiment, a processor of the MPS other than the processor 18 is configured to make the P&O calculations. In yet another exemplary embodiment wherein the system 10 is not part of the MPS but rather is used in conjunction with the MPS, a processor within the MPS is configured to make the P&O calculations. In an embodiment wherein the processor 18 is not configured to make the P&O calculations, the processor 18 is electrically connected to the processor making the P&O calculations, and is configured to receive the calculations from that processor. In an exemplary embodiment, the electrical connections between the sensors 14 and the processor making the P&O calculations, and, if appropriate, the electrical connections between multiple processors, may be made through one or more wires. However, those of ordinary skill in the art will appreciate that this connection may be made using any number of electrical connection techniques, including hardwired and wireless connections.

The medical device 16 may be a device or tool, such as, for example, a catheter, that is suitable for use and operation in a magnetic field environment, such as that generated by the magnetic field generator/transmitter assembly 12. The medical device 16 is configured for use in connection with the performance of one or more medical procedures or activities (e.g., mapping, imaging, navigation, therapy delivery, diagnostics, etc.). The positioning sensors 14 may be mounted to, or otherwise disposed within or on, the medical device 16. In an exemplary embodiment illustrated in FIG. 3a wherein the medical device 16 is a catheter having a proximal end 24 and a distal end 26, one or more of the positioning sensors 14 are disposed at or near the distal end 26 of the catheter, as well as at any other suitable position in or on the medical device 16.

In an embodiment wherein the system 10 comprises a MPS, the processor 18 may be configured to perform or carry out a number of functions. For example, as at least briefly described above, the processor 18 may be configured to be responsive to the positioning signals 22 generated by the positioning sensors 14 to calculate respective P&O readings for each sensor 14. Because each sensor 14 continuously generates the corresponding positioning signal 22 when disposed within the magnetic field, the P&O of the sensor 14 is continuously calculated and recorded by the processor 18. Thus, one function performed by the process 18 is real-time tracking of each positioning sensor 14, and therefore, the corresponding medical device(s) 16, in three-dimensional space.

As will be described in greater detail below, another function the processor 18 may be configured to perform is computing or determining the contour or reconstructing the shape of the medical device 16 based, at least in part, on the positioning information of the sensors 14 associated with the medical device 16. The computed contour or shape may be used for a number of purposes such as tracking the position and movement of the medical device 16.

Yet another function that the processor 18 may be configured to perform is the mapping of a volume within a region of interest (ROI) of a patient's body (e.g., an organ such as the heart) using the real-time positioning information determined as described above and to be described below.

Alternatively, the system 10 may include multiple processors wherein a first processor is configured to determine the positioning information, while a second separate and distinct processor (i.e., processor 18) is configured to compute or determine the contour or shape (hereinafter collectively referred to as “contour”) of the medical device 16 or to perform the mapping function to be described below. Similarly, in an embodiment wherein the system 10 is separate and distinct from the MPS but configured for use in conjunction to the MPS, a processor within the MPS is configured to determine the positioning information, while the processor 18 of the system 10 is configured to perform the contour computation/determination or mapping functions. In either of the latter two embodiments, the processors can be configured to communicate with each other such that the positioning determinations made by the first processor would be transmitted to and received by the second processor (i.e., the processor 18) for use in the computation of the contour of the medical device 16 and in the mapping operation. Therefore, while the description below is limited to the embodiment wherein the processor 18 is part of the MPS and performs the functions described above, it is not meant to be limiting in nature.

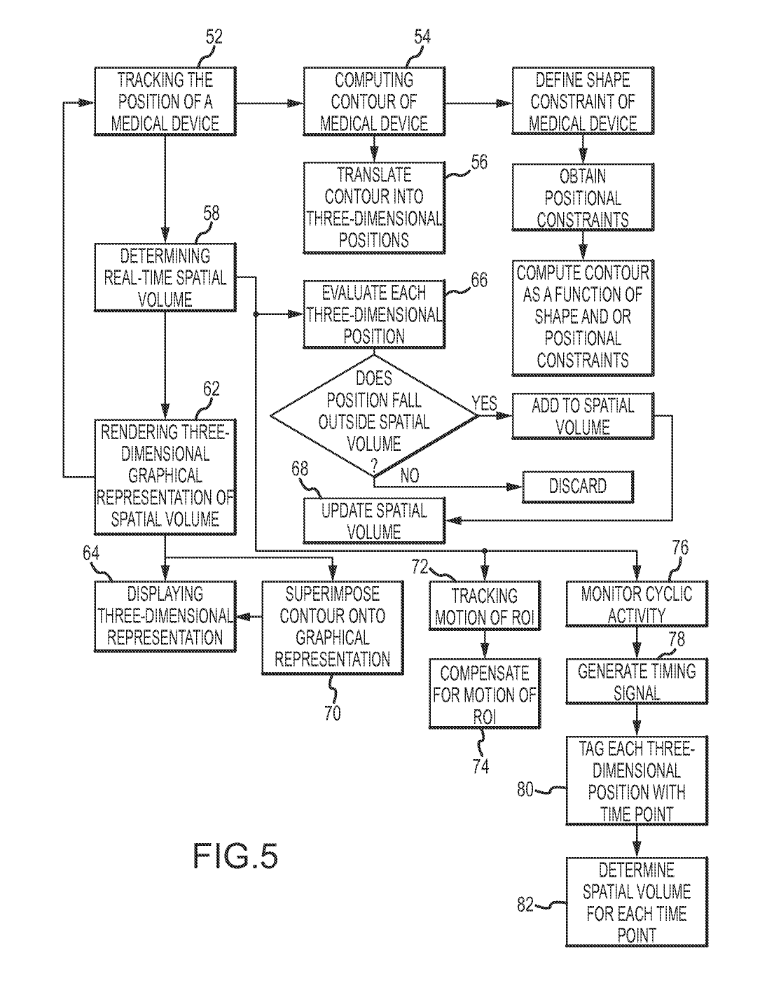

Accordingly, in an exemplary embodiment, and with reference to FIG. 5, the processor 18 is configured to compute a contour of the medical device 16 (step 54 in FIG. 5), and to then, based on the computed contour, map a volume within a ROI (e.g., the ROI 21 illustrated in FIG. 3b) located in a patient's body (step 62 in FIG. 5). More particularly, and as will be described in greater detail below, the processor 18 is configured to continuously track the position of a plurality of positioning sensors 14 associated with the medical device 16, and to then compute/determine a contour or reconstruct a shape of the medical device 16 based, at least in part, of the positioning information (P&O) corresponding to the positioning sensors 14. Once the contour is computed, the processor 18 is further configured to map a volume within the ROI 21 using the known P&O information corresponding to the positioning sensors 14 and the calculated P&O information corresponding to a plurality of virtual points 28 disposed along the computed contour of the medical device 16 (best shown in FIG. 3a).

FIG. 3b is a diagrammatic view of a computed contour 30 produced by the processor 18 representing the shape of the medical device 16 illustrated, for example, in FIG. 3a. One exemplary embodiment that may be used to compute the contour or to reconstruct the shape of the medical device 16 is that described in U.S. Provisional Patent Application Ser. No. 61/291,478 filed on Dec. 31, 2009 and entitled “Tool Shape Estimation,” which is incorporated herein by reference in its entirety. To summarize, the processor 18 is configured to compute the contour of the medical device 16 as a function of positional and/or shape constraint(s). More particularly, the algorithm for computing the contour may include inputs corresponding to one or more positional constraints, which may include the device's current location from one or more positioning sensors 14, as well as one or more locations corresponding to points along the path where the patient's body anatomically constrains free movement of the device in at least one degree of freedom. The positional constraints may also include locations of other anatomically constricting landmarks and the like. The algorithm may further include inputs corresponding to one or more shape constraints, which may include, for example, a relaxation shape of the medical device 16, as well as dimensions, pre-curves, and the like of the device 16.

As briefly mentioned above, one positional constraint that may be used to compute the contour of the medical device 16 is the “current” location of the device 16 as determined by the positioning sensors 14. In an exemplary embodiment, the processor 18 collects real-time sensor locations or P&O calculations (also referred to herein as “data point 32” or “data points 32”) for each positioning sensor 14. Since the positioning sensors 14 are physically associated with the medical device 16, their locations, and therefore, the locations of the portions of the device 16 at which the sensors 14 are disposed, are known. These known data points 32 (32₁, 32₂, 32₃, . . . , 32_{N}), which are disposed at different points along the medical device 16, may be used by the processor 18 in the computation of the contour of the medical device 16.

Another type of positional constraint that may be taken into account in computing a contour are anatomical constraints. More particularly, knowing where the medical device 16 is disposed within the patient's anatomy (e.g., heart), and/or knowing the route the medical device 16 took to get there, the locations of various anatomical landmarks that restrict or constrain the position and/or movement of the medical device 16 may be known. For example, and as illustrated in FIGS. 3a and 3b, if the medical device 16 travels through and/or is disposed within the Inferior Vena Cava (IVC) 34 and/or the fossa ovalis 36, for example, the regions proximate those anatomical structures anatomically constrain the portions of the medical device 16 passing therethrough. Accordingly, in this particular example, positional constraints are the locations of the IVC 34 and the fossa ovalis 36. The locations of these anatomical landmarks may be determined and recorded using a positioning sensor 14 when the device 16 passes through landmarks. The recorded locations define positional constraints on the contour computation that the processor 18 may factor into the contour computation. While the IVC 34 and the fossa ovalis 36 are specifically identified as positional constraints, it will be appreciated that other anatomical structures may constitute positional constraints. For example, in the illustrated depicted in FIGS. 4a and 4b, the Superior Vena Cava (SVC) 37 is a positional constraint. Accordingly, the present invention is not limited to any particular anatomical positional constraints.

In an exemplary embodiment, the locations of the anatomical structures or landmarks that define positional constraints may be determined through interaction with a user. The user may visually detect (e.g., according to an inspection of an x-ray image of the region of interest 21) when the device 16 passes through the anatomically constraining location in the body, such as, for example, the IVC 34, and more particularly when a part of the medical device 16 (e.g., the tip) passes through the IVC 34. In an exemplary embodiment, the system 10 includes a user input device 38 (shown in FIG. 1) electrically connected to and configured for communication with the processor 18. The user input 38 may take the form of a button, a switch, a joystick, a keyboard, a keypad, a touch screen, a pointing device (e.g., mouse, stylus and digital tablet, track-ball, touch pad, etc.), and the like. When the user detects passage through the IVC 34, the user may command the processor 18 through the user interface 38 to record the current position of the positioning sensor 14 that is currently disposed at the location of the IVC 34. The processor 18 then records the location as detected by the positioning sensor 14 as a data point 32. Accordingly, the current locations of the positioning sensors 14, and the locations of the constraining anatomical structures (represented as data points 32) collectively define a set of positional constraints used in the contour computation. Accordingly, in an exemplary embodiment, one or more positional constraints, including, for example, locations of positioning sensors 14 associated with the medical device 16, and/or location(s) corresponding to anatomical constraining location are obtained by the processor 18.

As described above, in additional to positional constraints, shape constraints of the medical device 16 may also be taken into consideration in the contour computation. The shape constraints correspond to the known mechanical characteristics of the medical device 16. For example, the processor 18 may know dimensions of the medical device, distances between positioning sensors 14, shapes, or pre-curves, and/or relaxation shape(s) of the device 16 and the like. Taking the relaxation shape as an exemplary constraint, the relaxation shape of the device 16 is predetermined and defined by a model stored in a storage medium 40 that is part of, and/or accessible by, the processor 18. The model may reflect a mathematical description of the curve (e.g., in the form of a polynomial expression) that corresponds to the relaxation shape of the device 16. For example, for a fixed shape catheter whose relaxation shape is defined in a Y-Z coordinate plane, such a relaxation shape model may define a Z-axis value for a given Y-axis value using, for example, a polynomial expression like z=ay²+by+c, where a, b, and c are coefficients (i.e., this assumes a second order polynomial—of course, higher order polynomial expressions are possible, as are other models employing different mathematical descriptions). It should be understood that the relaxation shape may be described in three-dimensions as well, and that the above description referenced to a two-dimensional mathematical description is exemplary only and not meant to be limiting in nature.

In another embodiment, the model may alternatively be configured to accommodate non-fixed shape tools, such as, for example, a steerable catheter. In such an alternate embodiment, however, the model may be configured to require the input of additional pieces of information, such as the location(s) of one or more restricting landmark(s) in close proximity to the device tip, and/or one or more location(s) from one or more additional positioning sensors 14 fitted to the non-fixed shape tool. Accordingly, one or more shape constraints are defined and provided to the processor 18.

Using some or all of the positional and shape constraints described above, a contour 30, such as that illustrated in FIG. 3b, is computed for the corresponding medical device 16 (step 54 in FIG. 5). In an exemplary embodiment, the contour computation may be carried out using spline interpolation. In any event, in one exemplary embodiment, the processor 18 computes the contour 30 by processing the input information corresponding to the shape constraint(s) and/or the positional constraint(s), and converges upon a solution consistent with the positional and shape constraints. The contour 30 represents the current shape or contour of the device 16.

Once the contour 30 of the medical device 16 is computed, the processor 18 is configured to translate it into a series of three-dimensional positions (P&Os and/or corresponding data points 32) (step 56 in FIG. 5). More particularly, the positions of various virtual points 28 on the contour 30 (shown in FIG. 3a) may be calculated based on the known P&O of the one or more of the positioning sensors 14 and, in certain embodiments, one or more of the shape and positional (e.g., anatomical, for example) constraints or information described above. As with the position of each sensor 14, the position of each virtual point 28 is recorded as a data point 32 (see FIG. 3b).

After the contour 30 of the medical device 16 is translated into a series of three-dimensional positions (P&Os and/or corresponding data points 32), a collection of data points 32 corresponding to both the P&Os of the positioning sensors 14 and the calculated P&Os of the virtual points 28 are evaluated to determine an initial spatial volume 42 (step 58 in FIG. 5). More particularly, each data point 32 is evaluated to determine whether it will be included in the boundary of the volume being modeled or mapped, or whether it will be discarded as being disposed within the interior of the volume. Once the outermost data points 32 are identified, the initial spatial volume 42 may be determined based on those outermost data points 32. The collection and/or evaluation of the data points 32, and/or the determination of the spatial volume 42 from a given set of internal locations may be carried out using known techniques. Examples of these known techniques may include, but are not limited to, those employed in the Carto™ system (a magnetic field-based system), commercially available from Biosense Webster and as generally described in U.S. Pat. Nos. 6,498,944 entitled “Intrabody Measurement” and 6,788,467 entitled “Medical Diagnosis, Treatment, and Imaging System,” each of which are incorporated herein by reference in their entireties, and the EnSite NavX™ system (a non-magnetic field-based system) referred to and incorporated by herein by reference above. Additionally, it will be understood that in an exemplary embodiment, as each data point 32 is calculated or determined, it is at least temporarily recorded and stored in a storage medium that is part of the processor 18, or a storage medium that is accessible by the processor 18, such as the storage medium 40 described above. If a data point 32 is determined to contribute to the boundary of the spatial volume 42, that particular data point 32 may be retained in the storage medium 40 as a contributing spatial point, and used to generate the spatial volume 42. If a data point 32 is determined to not contribute to the boundary of the spatial volume 42, it may be discarded from the storage medium 40 or recorded as a non-contributing spatial position, for example.

Once the initial spatial volume 42 is determined, it may be updated as new P&O information and corresponding data points 32 are collected. More particularly, as the medical device 16 is swept or moved about the volume being modeled (i.e., the ROI 21), a plurality of subsequent positions (P&O) of the positioning sensors 14 and the virtual points 28 are determined or calculated, and data points 32 corresponding to each P&O are recorded. As each data point 32 or set of data points 32 are collected, the processor 18 is configured to unify or apply the new data point(s) 32 to the evolving spatial volume 42. More particularly, for each individual new data point 32, the processor 18 determines whether the data point 32 will contribute to the volume boundary. For example, if the processor 18 determines that the data point 32 falls or is located outside of the currently determined spatial volume 42, it will further determine that that particular data point 32 will be used to update the spatial volume 42. Alternatively, if the processor 18 determines that the data point 32 corresponds to a location residing within the determined spatial volume 42, it will be further determined that that particular data point 32 will not be contributing to the volume boundary.

If the evaluated data point 32 is determined to correspond to a position within the determined spatial volume, the data point 32 may be discarded or recorded as, for example, a non-contributing spatial position disposed within the generated spatial volume 42. On the other hand, if the data point 32 falls or is located outside of the current determined spatial volume 42 such that it will be used to update the spatial volume 42, the processor 18 is configured to update the previously determined spatial volume 42 by determining a new, updated spatial volume 42 to broaden the envelope of the spatial volume 42 to include the new position(s) (i.e., data point(s) 32) (e.g., the determined spatial volume 42 is revised to include the three-dimensional positions falling outside of the previously determined real-time spatial volume). Accordingly, as the spatial volume 42 grows, it becomes more accurate as new positions (i.e., P&O calculations/data points 32) are added thereto.

In addition to the functionality described above, in an exemplary embodiment, the processor 18 is further configured to render a real-time three-dimensional graphical representation 44 of the surface of the determined spatial volume 42 (step 62 in FIG. 5). More particularly, the processor 18 is configured to apply known three-dimensional rendering techniques to the determined real-time spatial volume 42 to render a real-time three-dimensional surface map or model 44 of the spatial volume 42. Additionally, as the determined spatial volume 42 is updated as subsequent or additional P&O calculations are made, the processor 18 is configured to render an updated three-dimensional graphic representation 44 of the updated spatial volume 42 so as to allow for an accurate real-time three-dimensional representation of the determined spatial volume 42. Accordingly, the three-dimensional representation 44 of the spatial volume 42 is a dynamic rendering in that it may be continuously updated as the medical device 16 continues to move within the ROI 21.

One well-known exemplary technique that may be applied to render the three-dimensional graphic representation 44 is the marching cube technique in which a polygonal mesh representing the volume being explored will be generated from the set of data points 32 forming the spatial volume 42. It should be noted that the marching cube technique is provided for exemplary purposes only and is not meant to be limiting in nature. In other exemplary embodiments of the system 10, other techniques or methods now known or hereinafter developed for rendering or generating a three-dimensional graphical representation of a determined spatial volume may be used to perform the same function. Therefore, these techniques/methods remain within the spirit and scope of the present invention.

As will be described in greater detail below, once the three-dimensional map/model 44 is rendered, it may be used by the processor 18 or other processor(s) for a number of different purposes, such as, for example and without limitation, mapping of electrophysiological data, mapping for use in locating the ostium of a vessel during a cannulation process, to aid in the navigation of medical devices/tools, and in many other ways now known or hereinafter developed in the art. In addition, as is well known in the art, the three-dimensional graphical representation 44 may be superimposed onto a previously acquired or real time image, such as, for example, a fluoroscopic two-dimensional image.

In an exemplary embodiment, the processor 18 may be still further configured to control a display device 20 (shown in FIG. 1) to cause the rendered three-dimensional surface map/model 44 to be displayed for the user of the system 10 to see (step 64 in FIG. 5). More particularly, in one exemplary embodiment, the processor 18 is configured to display, in two-dimensions, an isometric representation of the generated three-dimensional map/model 44 of the determined spatial volume 42. Accordingly, the processor 18 is electrically connected to the display device 20 so as to communicate the three-dimensional map/model 44 for display on the display device 20. In an exemplary embodiment, the display device 20 takes the form of a display monitor, such as, for example, a computer monitor. It should be noted, however, that other types of display devices configured to visually display the three-dimensional surface map/model 44 may also be used, and therefore, remain within the spirit and scope of the present invention. Alternatively, in other exemplary embodiments, a processor other than the processor 18 that is electrically connected to, and in communication with, the processor 18 may be configured to control the display device 20 to display the three-dimensional map/model 44 that is rendered or generated by the processor 18 (as opposed to processor 18 being configured to control the display device 20). In another exemplary embodiment, the processor 18 may be further configured to display or render the three-dimensional representation 44 in a two-dimensional space. More particularly, once a line of sight is defined, the three-dimensional representation 44 can be rendered in two-dimensions by rendering only the visualized parts of the volume determined by the first pixel/voxel that the defined line of sight encounters on the volume.

With continued reference to FIG. 5, in an exemplary embodiment, the processor 18 may be yet still further configured to superimpose a graphical representation of the medical device 16 (i.e., its computed contour) onto the three-dimensional graphic representation 44 of the spatial volume 42 (step 70 in FIG. 5), and to then display the composite image on, for example, the display device 20. In another exemplary embodiment, another processor within the system 10 or the MPS may be configured to perform this function. Because the medical device 16 is used to create the spatial volume 42 which is then rendered as the graphical representation 44, it is inherently registered to the volume/representation and can therefore be rendered within the representation 44. The contour of the medical device 16 may be superimposed using techniques such as those described in one or more of, for example, U.S. Patent Application Ser. No. 61/291,478 filed on Dec. 31, 2009 and entitled “Tool Shape Estimation;” U.S. Pat. No. 6,233,476 entitled “Medical Positioning System;” U.S. Pat. No. 7,343,195 entitled “Method and Apparatus for Real Time Qualitative Three-Dimensional Image Construction of a Moving Organ and Intra-Body Navigation;” U.S. Patent Publication No. 2004/0254437 entitled “Method and Apparatus for Catheter Navigation and Location and Mapping in the Heart;” and U.S. Patent Publication No. 2006/0058647 entitled “Method and System for Delivering a Medical Device to a Selected Position within a Lumen,” each of which is incorporated herein by reference in its entirety.

More particularly, in an exemplary embodiment, the processor 18 is configured to determine a location in a reference coordinate system of the computed contour 30 of the medical device 16. The reference coordinate system may be the coordinate system of the MPS, and the location of the contour may be determined using, for example, the positioning information (P&O) described above corresponding to the positioning sensors 14. The location may be determined after the contour computation, or as a unitary process with the contour computation. Once the location is determined, the processor 18 is configured to generate a graphical representation of the computed contour 30, and to superimpose it onto the graphical representation 44.

In addition to the description above, in an exemplary embodiment, the system 10, and the processor 18, in particular, is configured to take into account one or more factors when determining the spatial volume 42 in order to increase the accuracy of the rendered three-dimensional model or map 44. For example, the processor 18 may be configured to take into account the resolution of the positioning (i.e., P&O) calculations (i.e., the smallest discernable distance between adjacent positions), the size of the position determinations, and the accuracy level of the position calculations.

An additional factor the processor 18 may be configured to take into account is the motion of the ROI 21, and/or the compensation for such motion. This motion may result from, for example, cardiac activity, respiratory activity, and/or from patient movements, and each type of movement must be accounted for. Accordingly in an exemplary embodiment, the system 10, and the processor 18, in particular, is configured to monitor the motion of the ROI 21, and to then compensate for that motion in the P&O calculations, the determination of the spatial volume 42, and/or the rendering of the three-dimensional model/map 44.

The concept of motion compensation is generally known in the art as seen, for example, by reference to U.S. Pat. No. 7,386,339 entitled “Medical Imaging and Navigation System,” which is incorporated herein by reference in its entirety. Reference is also made to U.S. patent application Ser. No. 12/650,932, filed Dec. 31, 2009 and entitled “Compensation of Motion in a Moving Organ Using an Internal Position Reference Sensor,” and U.S. Provisional Patent Application Ser. No. 61/291,478 filed Dec. 31, 2009 and entitled “Tool Shape Estimation,” each of which is incorporated herein by reference in its entirety. Accordingly, the motion compensation functionality may be carried out as described in one or more of the aforementioned patents or patent applications. Therefore, only a brief and general explanation of motion compensation will be provided here.

In one an exemplary embodiment wherein the system 10 is configured to compensate for motion of the ROI 21 and other motion that may impact the device tracking, the system 10 includes, for example, a sensor 46, such as a patient reference sensor (PRS), ECG patches, and the like, that is/are attached to the body of the patient to provide a stable positional reference of the patient's body so as to allow motion compensation for gross patient body movement and/or respiration induced movements. For clarity purposes, this sensor will hereinafter be referred to as patient reference sensor 46 or PRS 46, for short. In this regard, the PRS 46 may be attached to the patient's manubrium sternum, a stable place on the chest, or other location that is relatively positionally stable. The PRS 46 is configured to detect movements (e.g., C-arm movements, respiration chest movements, patient movements, etc.) that may impact the integrity of the device tracking. More particularly, the PRS 46 is configured to detect one or more characteristics of the magnetic field in which it is disposed, and the processor 18 is configured to provide a location reading (i.e., P&O) based on the output of the PRS 46 indicative of the PRS's three-dimensional position and orientation in the reference coordinate system. Accordingly, using the PRS 46 the processor 18 is configured to continuously record signals indicative of the motion of the region of interest 21, and to analyze the signals. Based on these signals, and the analysis thereof, the processor 18 may modify the P&O calculations/determinations for the positioning sensors 14 and/or the virtual points 28 to adjust the location of the medical device 16 that is based on these P&O calculations/determinations.

In addition to the factors described above that processor 18 may take into account in determining the spatial volume 42 and the graphical representation 44, the processor 18 may be further configured to employ time-dependent gating in an effort to increase accuracy of the determined spatial volume 42, and in the three-dimensional representation 44 thereof. In general terms, time-dependent gating comprises monitoring a cyclic body activity, such as, for example, cardiac or respiratory activity, and generating a timing signal, such as an organ timing signal, based on the monitored cyclic body activity. One reason for employing such a procedure is that as the medical device 16 (and therefore, the positioning sensors 14 thereof) move about the ROI 21, data points 32 (i.e., P&O determinations/calculations) are collected at all stages of the cyclic activity without regard to the phase of the activity. One example is the cardiac cycle or heart beat. Since the heart (i.e., the ROI 21) changes shape throughout the cardiac cycle, and since data points 32 are collected at all stages of the cardiac cycle, not all of the collected data points 32 will correspond to the same “shape” or “size” of the heart (i.e., ROI 21). Therefore, if a spatial volume is determined using all of the data points 32 without regard to the point in the cardiac cycle at which each data point 32 was collected, the resulting spatial volume will be inaccurate. In other words, without filtering out the data points such that only data points 32 for a particular phase or point in the cardiac cycle are used in determining the spatial volume 42 for that particular phase of the cardiac cycle, an accurate spatial volume cannot be determined. Accordingly, in an exemplary embodiment, the present invention provides for phase-based data point/location collection, which enables the determination of one or more spatial volumes 42 in accordance with phase, and therefore, the rendering of one or more three dimensional graphical representations 44 of the determined spatial volume(s) 42 in accordance with phase. This allows for a more realistic representation of the changing volume of the ROI 21 (e.g., a heart chamber) as it changes throughout the different phases of the cyclic activity (e.g., the cardiac cycle).

Accordingly, in an exemplary embodiment, the system 10 includes a mechanism to measure or otherwise determine a timing signal of the ROI 21, which, in an exemplary embodiment, is the patient's heart, but which may also include any other organ that is being evaluated. For purposes of clarity, however, the following description will be limited to an ROI 21 that comprises the patient's heart. The mechanism may take a number of forms that are generally known in the art, such as, for example, a conventional electro-cardiogram (ECG) monitor. A detailed description of a ECG monitor and its use/function can be found with reference to U.S. patent application Ser. No. 12/347,216, filed Dec. 31, 2008 and entitled “Multiple Shell Construction to Emulate Chamber Contraction with a Mapping System,” which is incorporated herein by reference in its entirety.

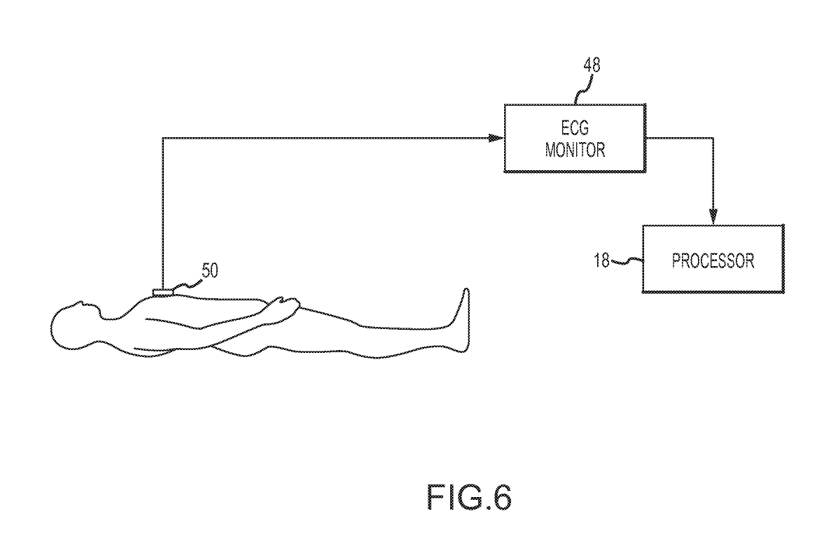

With reference to FIG. 6, in general terms, an ECG monitor 48 is provided that is configured to continuously detect an electrical timing signal of the patient's heart through the use of a plurality of ECG electrodes 50, which may be externally-affixed to the outside of a patient's body. The timing signal generally corresponds to the particular phase of the cardiac cycle, among other things. In another exemplary embodiment, rather than using an ECG to determine the timing signal, a reference electrode or sensor positioned in a fixed location in the heart may be used to provide a relatively stable signal indicative of the phase of the heart in the cardiac cycle (e.g., placed in the coronary sinus). In still another exemplary embodiment, a medical device, such as, for example, a catheter having an electrode may be placed and maintained in a constant position relative to the heart to obtain a relatively stable signal indicative of cardiac phase. Accordingly, one of ordinary skill in the art will appreciate that any number of known or hereinafter developed mechanisms or techniques, including but not limited to those described above, may be used to determine a timing signal of the ROI 21.

Once the timing signal, and therefore, the phase of the patient's heart, is determined, the data points 32 (i.e., P&O determinations/calculations) corresponding to the position of the positioning sensor 14 and other virtual points 28 on the contour of the medical device 16 may be segregated or grouped into a plurality of sets based on the respective phase of the cardiac cycle during (or at which) each data point 32 was collected. Once the data points 32 are grouped, the processor 18 is configured to determine a respective spatial volume 42 for one or more phases of the cardiac cycle in the manner described above using only those P&O calculations or data points 32 that were collected during that particular phase for which the spatial volume 42 is being determined. The processor 18 is further configured to render a corresponding three-dimensional representation 44 for each determined spatial volume 42 in the manner described above. Because the timing signal of the ROI 21 is known, as each subsequent P&O calculation is made, the data point 32 is tagged with a respective time-point in the timing signal and grouped with the appropriate previously recorded data points 32. The subsequent data points 32 may then be used to update, if appropriate, the determined spatial volume 42 for the phase of the cardiac cycle during which the data point 32 was collected, as well as the rendered three-dimensional graphical representation 44 of the determined spatial volume 42.

Once a three-dimensional graphical representation 44 is rendered for each phase of the cardiac cycle, the graphical representation 44 corresponding to the current phase of the timing signal may be presented to the user of the system 10 at any time. In an exemplary embodiment, the processor 18 may be configured to play-back the rendered three-dimensional graphical representations 44 (e.g., sequentially reconstructed and displayed on the display 20) in accordance with the real-time measurement of the patient's ECG. Therefore, the user may be presented with an accurate real-time three dimensional graphical representation 44 of the determined spatial volume 42 of an ROI 21 regardless of the phase of the cardiac cycle. Accordingly, it will be understood and appreciated that the spatial volume 42 and corresponding graphical representation 44 for each phase may be stored in a storage medium, such as, for example, the storage medium 40, that is either part of or accessibly by the processor 18 such that the processor 18 may readily obtain, render, and/or display the appropriate spatial volume 42 and graphical representation 44.

It should be noted that while the description above has been primarily directed to the use of time-dependent gating relating to a patient's cardiac activity, such a description has been provided for exemplary purposes only. In other exemplary embodiments, other cyclic activities may be monitored and taken into account in a similar manner to the monitoring and the taking into account of the cardiac cycle. Accordingly, the present invention is not meant to be limited to only time-dependent gating of a patient's cardiac activity.

While the description thus far has been primarily with respect to an embodiment wherein the system 10 comprises a MPS, in another exemplary embodiment briefly described above with respect to FIG. 2, the system 10 is not part of a MPS but rather is separate and distinct system that is used in conjunction with a MPS. Because this embodiment of the system 10 is separate and distinct from the MPS, it does not necessarily include all of the components of the embodiment wherein the system 10 comprises the MPS, such as, for example, the magnetic field generator 12. Accordingly, in this embodiment, the processor 18 may be electrically connected to (via wires or wirelessly), and configured for communication with, the MPS such that the P&O calculations made or determined by the MPS may be communicated to the processor 18. Other than not comprising all of the same components as the embodiment described above, the system 10, and the processor 18, in particular, function and operate in the same way as described above. Accordingly, the description set forth above relating to the function and operation of the system 10 applies here with equal force, and therefore, will not be repeated. Thus, in view of the above, the system 10 may take on any number of different arrangements and compositions, all of which remain within the spirit and scope of the present invention.

With reference to FIG. 5, it will be appreciated that in addition to the structure of the system 10, another aspect of the invention in accordance with the present teachings is a method of three-dimensionally mapping or modeling a volume within a ROI located within a body. In an exemplary embodiment, a first step 52 includes tracking the position of the medical device 16 within the ROI 21 in real-time. In an exemplary embodiment, this tracking step 52 is performed by a first substep 54 of computing a contour of the medical device 16 as a function of at least one of a positional constraint and shape constraint; and a second substep 56 of translating the contour into a series of three-dimensional positions (P&Os) corresponding to various locations on the contour of the device 16. The three-dimensional positions/locations comprise both known and virtual positions, and the virtual positions are translated based on a shape and/or space constraint.

A second step 58 includes determining a real-time spatial volume 42 based on the three-dimensional positions calculated/determined in the first step 52, including at least one virtual three-dimensional position. In an exemplary embodiment, the spatial volume 42 is determined based on one or more of the translated three-dimensional positions, as well as at least one previously acquired/recorded three-dimensional position. In an exemplary embodiment, the tracking step 52 may further include the substep of recording the three-dimensional positions as spatial positions or data points 32; and the determining step 58 may include determining the spatial volume 42 using the recorded data points 32.

A third step 62 includes rendering a real-time three-dimensional graphical representation 44 of the spatial volume 42 that was determined in the second step 58.

In an exemplary embodiment, the method may also include a fourth step 64 comprising controlling a display device 20 to cause the three-dimensional graphical representation 44 to be displayed on the display device 20.

In an exemplary embodiment, the aforedescribed steps are continuously repeated until the methodology is stopped. Accordingly, the tracking step 52, and the translating substep 56 described above, in particular, comprises translating the contour into a first set of three-dimensional positions. As the tracking step 52 is repeated, one or more subsequent contours of the medical device 16 are computed in the same manner described above, and those contours are translated into respective sets of three-dimensional positions in the same manner described above. As each set of three-dimensional positions are collected and recorded, the determining step 58 includes a substep 66 of evaluating each three-dimensional position to determine whether the position is within or outside of the previously determined spatial volume 42. In a substep 68, the spatial volume 42 is updated if at least one of the recorded three-dimensional positions fall outside of the previously determined spatial volume 42 to include the at least one three-dimensional position (i.e., the data points 32 corresponding to those positions) (e.g., the determined spatial volume 42 is revised to include the three-dimensional positions falling outside of the previously determined real-time spatial volume).

The method may still further include a fifth step 70 comprising generating a three-dimensional graphical representation of the computed contour 30 of the medical device 16, and superimposing the same onto the three-dimensional graphical representation 44 of the spatial volume 42.

In an exemplary embodiment, the method yet still further includes a sixth step 72 comprising tracking the motion of the ROI 21, or other motion that may impact the tracking of the medical device 16, over time; and a seventh step 74 of compensating for such motion in the P&O calculations/determinations, the determination of the spatial volume 42, and/or the three-dimensional graphical representation 44 of the spatial volume 32.

The method may still further an eighth step 76 of monitoring a cyclic body activity occurring within the ROI 21. In such an embodiment, the method further includes a ninth step 78 of generating a timing signal based on the monitored cyclic body activity, and a tenth step 80 of tagging each P&O determination (i.e., data point 32) with a respective time-point in the timing signal. When the method includes these two steps, the determining a spatial volume step (step 58) may include a substep 82 of determining a respective spatial volume 42 for one or more time-points in the timing signal; and the rendering a three-dimensional graphical representation step (step 62) may include the substep of rendering a three-dimensional graphical representation for each respective spatial volume 42 corresponding to one or more time-points in the timing signal.

In accordance with another aspect of the invention, in an exemplary embodiment, once the three-dimensional graphical representation 44 of the determined spatial volume 42 within the ROI 21 is rendered, it may be used, for example, to map a surface of anatomical structure to which the three-dimensional representation 44 corresponds (i.e., located within the ROI 21). More particularly, and as will be described in greater detail below, the processor 18, or another processor that is part of or used in conjunction with the system 10, may be configured to superimpose, in real time, marks on the three-dimensional graphical representation 44 corresponding to areas of the anatomical structure that a medical device, such as, for example, the medical device 16, or some other suitable medical device, has contacted while performing a medical procedure. Using the contact points and corresponding marks, a real-time map of the surface may be constructed and superimposed onto the three-dimensional graphical representation 44.

One exemplary procedure with which this aspect of the invention finds particular application is a cannulation procedure. In such a procedure, a physician or clinician inserts a medical device or tool, such as, for example, a catheter, into an insertion region of a patient, which, in an exemplary embodiment, may comprise the superior vena cava (SVC) of the heart. Once the medical device or tool is inserted, the physician or clinician uses the device or tool to probe around a surface of the anatomical structure searching for the ostium of a vessel, for example. Procedures such as this can be rather lengthy procedures that employ a “trial and error” method for searching for the ostium (i.e., the physician “pokes” or probes around the surface until the ostium is found). Without information regarding where the physician previously poked or probed, the same area may be poked or probed several times, thereby lengthening the time of the procedure and increasing the amount of radiation exposure (i.e., x-ray) needed, thus resulting in exposing the anatomical structure or ROI 21 to redundant irritation.

As will be described in greater detail below, in accordance with this aspect of the invention, as the physician probes the surface of the anatomical structure and makes contact therewith, the points on the three-dimensional graphical representation 44 that correspond to the point or area of the surface of the anatomical structure that the medical device or tool contacted are marked. Using the marked points, a map of the probed surface may be generated to provide representations of areas that need not be revisited in the attempt to located the ostium of the vessel. The marked-up image and/or the resulting surface map may then be presented to the physician via a display device, such as, for example, the display device 20, or some other comparable display device, to allow the physician to see where s/he already probed or “poked” in searching for the ostium so that those areas may be avoided as s/he continues to probe. By providing the physician this information, redundant probing is eliminated, or at least substantially reduced, thereby shortening the procedure and reducing the likelihood of exposing the patient to potentially increased and unnecessary radiation (x-ray).

It should be noted that while in an exemplary embodiment the contact points may be marked or otherwise represented on the real-time three-dimensional graphical representation 44 rendered as described in great detail above, in other exemplary embodiments, a different real-time or previously acquired image or model of the anatomical structure may be obtained and the contact points may be marked thereon. This image or model may be a two- or three-dimensional image/model, for example, and may include, without limitation, a fluoroscopic image or an image or model generated using one or more different imaging/modeling modalities now known or hereinafter developed. Accordingly, one of ordinary skill in the art will appreciate that any number of images or models of the anatomical structure of interest may be used. In an effort to avoid confusion and to clearly illustrate that this aspect of the present invention is not limited to use with only the three-dimensional graphical representation 44, the image or model to be marked with contact points will hereinafter by referred to as “image 84.”

In an exemplary embodiment, the system 10 is configured to carry out the marking functionality described above. More particularly, in an exemplary embodiment, the processor 18 of the system 10 is configured to carry out the functionality. In another exemplary embodiment, however, the system 10 may include a processor other than the processor 18 that is configured to carry out some or all of the functionality. In such an embodiment, the two processors would be coupled together and configured to communicate with each other such that, for example, the image 84 rendered by the processor 18 may be communicated to the other processor (in the instance wherein the image 84 (e.g., the three-dimensional graphical representation 44) is rendered by the processor 18 is the image or model used). In yet another exemplary embodiment, a processor that is separate and distinct from the system 10, but configured to be used in conjunction with the system 10, may be configured to carry out some or all of the functionality (e.g., the marking functionality). As with the two-processor arrangement described above, in this embodiment the separate and distinct processor and the processor 18 would be coupled together and configured to communicate with each other. While many different arrangements may be implemented to carry out the aforedescribed functionality, for the sake of clarity and brevity, only the embodiment wherein the processor 18 is configured to carry out the marking functionality will be described in greater detail below. It should noted, however, that the present invention is not meant to be limited to such an implementation or arrangement, but rather other arrangements or implementations may be used to carry out the same functionality and remain within the spirit and scope of the present invention.

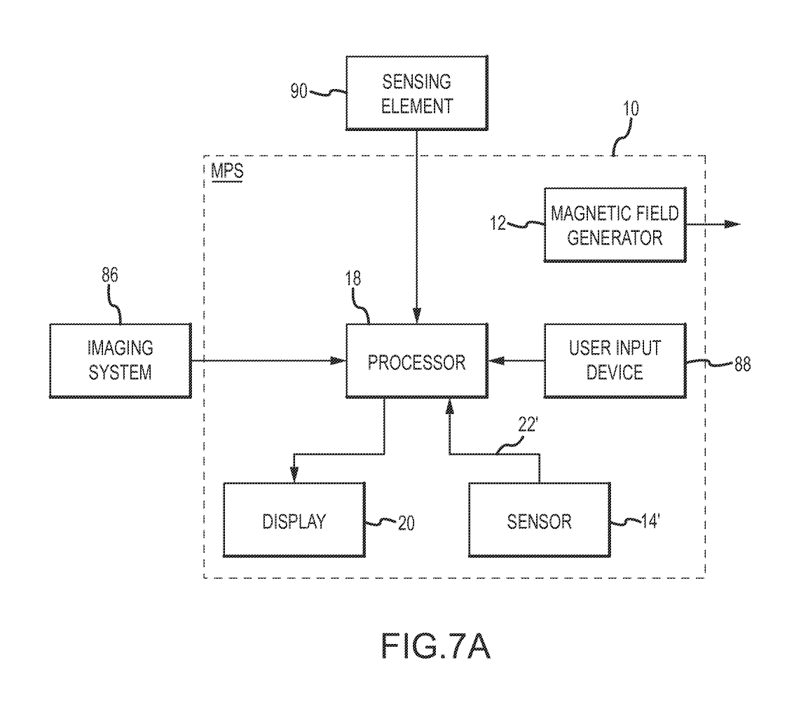

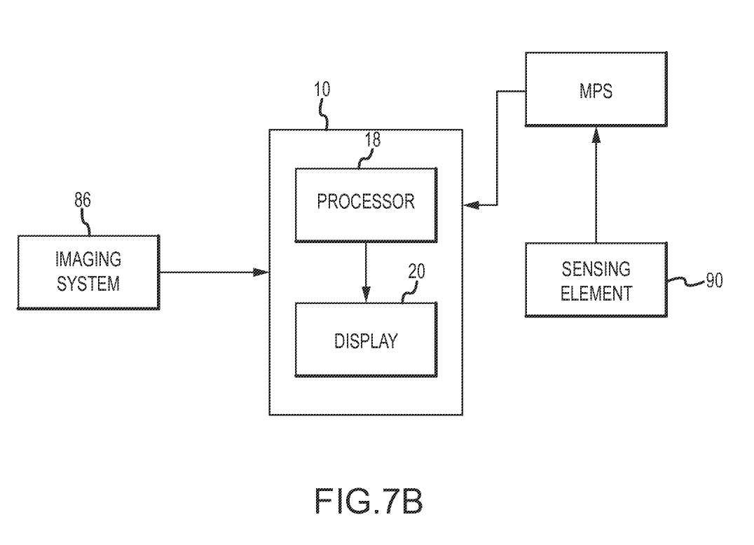

Accordingly, in an exemplary embodiment, the processor 18 is configured to obtain the image 84 of an anatomical structure of interest. As described above, the image 84 may be a real-time (e.g., a fluoroscopic image, three-dimensional representation 44, etc.) or previously acquired image (e.g., CT image, MRI image, previously generated model, etc.), and/or may be a two- or three-dimensional image. As described above, if the system 10, and the processor 18 in particular, is part of or used in conjunction with a MPS, the MPS may be configured to generate the image 84. Alternatively, as illustrated in FIGS. 7a and 7b, for example, the system 10 may include, or be coupled to and in communication with, an imaging system 86, such as, for example and without limitation, a fluoroscopic imaging system, configured to generate the image 84 of the anatomical structure of interest.

As briefly described above, in a cannulation process, a physician or clinician probes around the surface of the anatomical structure of interest searching for the ostium of a vessel. In an exemplary embodiment, the medical device or tool used is the medical device 16 described above. However, in another exemplary embodiment, a separate and distinct medical device is used. In the interest of clarity, the medical device or tool used with respect to this aspect of the invention will be referred to hereinafter as “medical device 16′.” In either instance, the processor 18 is configured to display where the medical device 16′ contacts the surface of the anatomical structure of interest.

With respect to determining the location of the medical device 16′ when contact occurs, the process described above with respect to determining positioning information of the positioning sensor(s) 14 of the medical device 16 may be used, and therefore, that description applies here with equal weight. Accordingly, the position of the medical device 16′ may be determined by using one of a number of different types of medical positioning systems (MPS), such as, for example, magnetic field-based or electric field-based MPS. For purposes of clarity and brevity, the following description will be limited solely to a magnetic field-based system such as that described above. Accordingly, in an exemplary embodiment, the medical device 16′ used in the cannulation procedure includes one or more positioning sensor(s) 14′ associated therewith for generating corresponding positioning signals 22′. For each of description purposes, the following description will be limited to an exemplary embodiment wherein the device 16′ includes a single positioning sensor 14′ generating a corresponding positioning signal 22′.

In the embodiment illustrated in FIG. 7a, the positioning signal 22′ is communicated to, and used by, the processor 18 to calculate a P&O of the positioning sensor 14′. In another exemplary embodiment, such as, for example and without limitation, the embodiment illustrated in FIG. 7b, the processor 18 may not be configured to calculate the P&O of the positioning sensor (i.e., the processor 18 is not part of a MPS). In such an embodiment, the P&O of the positioning sensor 14′ may be calculated by a processor of a MPS and then communicated to the processor 18, which may then use the P&O calculation as described above, and as will be described in greater detail below.

With respect to determining when the medical device 16′ contacts the surface of the anatomical structure, many different contact sensing techniques may be used. For example, using a real-time image, such as a fluoroscopic image, a physician may be able to visualize when the medical device or tool contacts the surface (i.e., tissue). In another example, a real-time image may be used in conjunction with a physician's tactile sensing to determine contact has been made. In either instance, when the physician believes contact has been made, he may trigger the calculation of a P&O by inputting a command into a user input device 88 coupled to, and configured for communication with, the processor 18 (or another processor that is configured to calculate the P&O of the medical device 16′), such as, for example, a keyboard, a joystick, a touch screen, a mouse, a button, a switch, and other like devices. Accordingly, the user input device 88 is configured to generate signal in response to an input by the user.

In another exemplary embodiment, the medical device or tool 16′ may have a sensing element 90 disposed at or near the tip thereof (i.e., at or near the distal end of the device 16′) and electrically connected to a processor, such as, for example and without limitation, the processor 18. The sensing element 90, which may comprise an electrode or a sensor, for example, is configured and operative to generate a signal indicative of contact between the sensing element 90 and the anatomical structure. Exemplary methods of contact sensing are described in U.S. patent application Ser. No. 12/347,216, filed Dec. 31, 2008 and entitled “Multiple Shell Construction to Emulate Chamber Contraction with a Mapping System,” incorporated herein by reference above. In an exemplary embodiment, the sensing element 90 may take the form of any one or more of a variety of electrical-based, electro-mechanical-based, force-based, optically-based, as well as other technology-based approaches known in the art for determining when the sensing element 90 is in contact with the surface of the anatomical structure.

An alternate approach for sensing contact is to assess the degree of electrical coupling as expressed, for example, in an electrical coupling index (ECI) between such a sensing element and the surface, as seen by reference to, for example, U.S. patent application Ser. No. 12/253,637, filed May 30, 2008 and entitled “System and Method for Assessing Coupling Between an Electrode and Tissue,” which is incorporated herein by reference in its entirety.

In yet another alternate approach, an electrically-measured parameter indicative of contact, such as, for exemplary purposes only, the phase angle of a measured complex impedance, may be used to determine when the sensing element 90 is in contact with tissue. One phase angle measurement may be as described in U.S. Patent Publication No. 2009/0171345 entitled “System and Method for Measurement of an Impedance Using a Catheter such as an Ablation Catheter,” which is incorporated herein by reference in its entirety.

When it is determined that the medical device 16′ has contacted the anatomical structure, the processor 18 is configured to calculate and record a corresponding P&O of the positioning sensor 14′ responsive to the indication of contact, and therefore, the signal generated by the sensing element 90. Accordingly, the processor 18 is configured to calculate the P&O of the positioning sensor 14′ at the time contact has been made to determine the location at which the contact occurred.

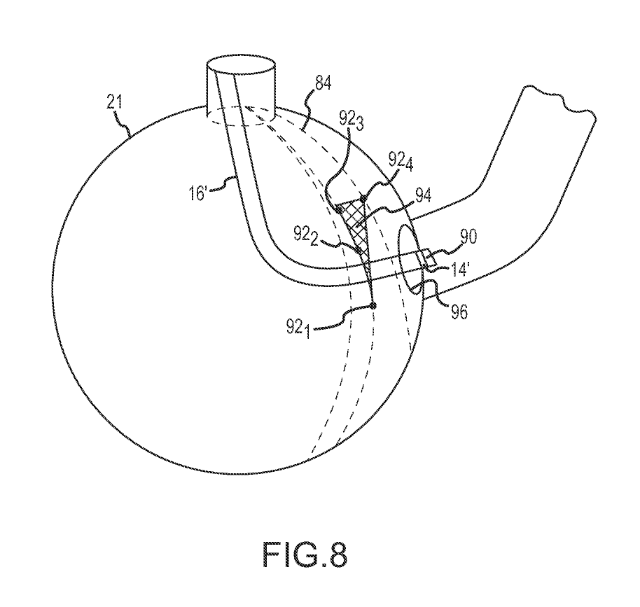

In an exemplary embodiment, and with reference to FIG. 8, the processor 18 is further configured to use the P&O calculation to superimpose a mark 92 onto the image 84 of the anatomical structure corresponding to the P&O of the positioning sensor 14′ of the medical device 16′ to indicate that the anatomical structure was contacted at the particular point on the anatomical structure where the mark 92 (i.e., 92₁, 92₂, 92₃, . . . , 92_{N}) was superimposed. The mark 92 may be graphically rendered in either two- or three-dimensions, and may include other content apart from the mark 92 itself. For example, in an exemplary embodiment, content such as time tags, time offset from a reference, textual comments, sensed electrical parameters, and the like may be included and displayed or stored with the mark 92. Each time the medical device 16′ contacts the surface, the process repeats, and the result is a collection of marks 92, such as that illustrated in FIG. 8, superimposed on the image 84 showing where the medical device 16′ has contacted the surface throughout the procedure.

In order to accurately display the marks 92 on the image 84, the coordinate system of the image 84 and that of the MPS that determines the P&O of the medical device 16′ when contact is made, may need to be registered with each other so that MPS location readings can be properly transformed into the image coordinate system of the particular image being used. When registration is required, once registered, a coordinate (i.e., position and orientation values) in one coordinate system may be transformed into a corresponding coordinate in the other coordinate system through the transformations established during the registration process, a process known generally in the art, for example as seen by reference to U.S. Patent Publication No. US2006/0058647 entitled “Method and System for Delivering a Medical Device to a Selected Position within a Lumen,” which is incorporated herein by reference in its entirety.

If the positioning information used for generating the image 84 is determined by an MPS other than the MPS used to determine the positioning information when contact is detected, or if the image 84 is not generated by an MPS at all, the two coordinate systems must be registered. Similarly, in another exemplary embodiment wherein the image 84 is generated using an imaging system or modality such as, for example, fluoroscopy, MRI, CT, or other now known or hereinafter developed imaging techniques, registration may be required.

If registration is required, the two coordinate systems may be registered using known registration techniques. For example, in an instance wherein the image is a fluoroscopic image, the coordinate system of the fluoroscopic image and that of the MPS may be registered by an optical-magnetic calibration process at installation. More particularly, the amplifiers of the MPS may be mounted on the C-arm of the fluoroscopic system, and therefore, the two coordinate systems will always be aligned. The MPS may include a reference sensor in the ROI 21 that identifies the amplifiers (and therefore the C-arm) movements, and in so doing, keeps track of the angulations of the fluoroscopic image at any given time. Other imaging systems or modalities may require several landmarks or fiducials to be pointed out. More particularly, a manual marking of fiducials or landmarks is required. If this registration technique is used in connection with a fluoroscopic image, the matching point need only be marked on the fluoroscopic image because the MPS, as described above, is registered with the C-arm and can map any marked fiducial to its own coordinate system. Accordingly, one of ordinary skill in the art will appreciate that any number of registration techniques exist that may be used to register the coordinate systems of the image and the system generating the positioning information for the medical device 16′, all of which remain within the spirit and scope of the present invention.

In an exemplary embodiment, the processor 18, or another processor within the system 10, or that is configured for communication with the system 10, may be further configured to generate a real-time surface map representing the cannulation area using the P&O calculations/data points corresponding to the marks 92 on the image 84. More particularly, the processor 18 may be configured to collect the P&O calculations corresponding to instances and locations when the medical device 16′ contacted the surface of the anatomical structure during the cannulation procedure. The processor 18 may be configured to then process the collection of P&O calculations and to generate a map or model 94 of the surface of the anatomical structure. The processor may be configured to process the collected P&O calculations and to generate the surface map 94 using any number of techniques known in the art. One such technique involves representing the surface with a polygonal mesh by generating a surface with a series of convex polygons (polyhedrals) that use the collected P&O calculations as vertices. As each new P&O calculation/data point is collected (i.e., each new P&O is calculated), the processor 18 is configured to update or “reconstruct” the surface map 94 to provide an accurate, real-time, surface map.

Once generated, the map or model 94 may be superimposed onto the image 84 or, alternatively, displayed separately, as will be described below. In either instance, the generated real-time surface map 94 may be used by the physician to determine which areas or regions of the anatomical structure, as opposed to simply individual points, have been probed, and therefore, need not be probed again, and which areas may still be probed in searching for the ostium (referred to in FIG. 8 and hereinafter as “ostium 96”). Accordingly, the generated surface map 94 may be used to aid the physician in converging toward the ostium 96 of interest.

In an exemplary embodiment, the processor 18 may be further configured to control a display device, such as, for example, the display device 20 or some other comparable display device, in order to cause the image 84 with the marks 92 superimposed thereon to be visually displayed. In addition to the marked-up image 84, the surface map 94 generated using the P&O calculations corresponding to the marks 92 may also be displayed on the display device 20, or another separate and distinct display device, either as being superimposed onto the image 84 or as a separate image. Alternatively, the processor 18 may be configured to communicate with another processor that, in turn, is configured to control a display device. In such an embodiment, the processor 18 would be electrically connected to the other processor and would be configured to transmit to the other processor the marked-up image 84 and or surface map 94 for display.

In an exemplary embodiment, the system 10, and the processor 18, in particular, may be further configured to represent the medical device 16′, and/or other medical devices or tools disposed within the ROI 21, on the image 84. In an embodiment wherein the image is generated using fluoroscopy, the representation of the medical device(s) or tool(s) may be part of the image by virtue of the fact that the fluoroscope generally visualizes or images the devices or tools that are disposed within the field of view of the fluoroscope. Alternatively, the representation may be generated using the positioning information (i.e., P&O) of the particular device or tool, and then superimposed onto the image 84. This latter approach may be carried out using the techniques described above with respect to generating a contour of the medical device 16 or reconstructing a shape of the medical device 16, and superimposing the contour/reconstruction of the medical device 16 onto the three-dimensional graphical representation 44 rendered by the processor 18, including the techniques described in, for example, U.S. Patent App. Ser. No. 61/291,478 filed Dec. 31, 2009 and entitled “Tool Shape Estimation,” incorporated herein by reference above Accordingly, this description will not be repeated here.

It should be noted that while the description above relating to contact sensing, P&O calculation upon contact detection, control of the display, generation of a surface map, etc. has been with respect to the processor 18 (i.e., the processor 18 being configured to carry out each of the functions), the invention is not intended to be so limited. Rather, in other exemplary embodiments, one or more processors that are either part of the system 10, or at least configured for communication with the system 10, may be used to carry out some of the functionality. For example, in an exemplary embodiment, a processor other than the processor 18 may be configured to detect when contact with the surface of the anatomical structure has been made, and to then communicate this occurrence to the processor 18, which may then calculate a P&O of the positioning sensor 14′ of the medical device 16′. This P&O may be used by the processor 18 to superimpose a mark 92 onto the image 84 or, alternatively, may be communicated to another processor that is configured to superimpose the mark onto the image. Accordingly, a variety of arrangements including one or more processors may be used to carry out the above described functionality, all of which remain within the spirit and scope of the present invention.

In addition to the description above, in an exemplary embodiment, the system 10, and the processor 18, in particular, is configured to take into account certain factors when, for example, determining the P&O of the sensor 14′ and superimposing the marks 92 on the image 84 in order to increase the accuracy of the calculations, markings, and surface map 94 constructed using the P&O calculations and marks 92. For example, the processor 18 may be configured to take into account the motion of the ROI 21, and/or the compensation for such motion. This motion may result from, for example, cardiac activity, respiratory activity, and/or from patient movements. Accordingly in an exemplary embodiment, the system 10, and the processor 18, in particular, is configured to monitor the motion of the ROI 21, and to then compensate for that motion in the P&O calculations, placement of the marks 92 on the image 84, and/or the construction of the surface map 94. As described above, the concept of motion compensation is generally known in the art, and therefore, the description above applies here with equal force and will not be repeated.

As also described above, another exemplary factor that processor 18 may take into account is time-dependent gating. In general terms, time-dependent gating comprises monitoring a cyclic body activity, such as, for example, cardiac or respiratory activity, and generating a timing signal (i.e., an organ timing signal) based on the monitored cyclic body activity. One reason for employing time-dependent gating is that as the positioning sensor 14′ moves about the ROI 21, P&O calculations are collected at all stages of the cyclic activity without regard to the phase of the activity. One example is the cardiac cycle or heart beat. Since the heart (i.e., the ROI 21) changes shape throughout the cardiac cycle, and since the P&O calculations or data points are collected at all stages of the cardiac cycle, not all of the collected data points will correspond to the same “shape” or “size” of the heart (i.e., ROI 21). Therefore, if all of the marks 92 are superimposed onto the image 84, or the surface map 94 is constructed using all of the data points without regard to the point in the cardiac cycle at which each data point was collected, the resulting marked-up image 84 and the surface map 94 will be inaccurate. In other words, without filtering out the data points such that only those data points for a particular phase or point in the cardiac cycle are used in superimposing the marks 92 onto the image 84 or in constructing the surface map 94, an accurate representation of prior contact with the surface and an accurate surface map cannot be constructed. Therefore, in an exemplary embodiment, the present invention provides for phase-based sensor location collection, which enables the marking of the image 84 to show points of contact and the construction of the surface map 94 in accordance with phase.

In an exemplary embodiment, the system 10 includes a mechanism to measure or otherwise determine a timing signal of the ROI 21, which, in an exemplary embodiment, is the patient's heart, but alternatively may include any other organ that is being evaluated. The description set forth above relating to the generation and use of the timing signal applies here with equal force, and therefore, will not be repeated. Therefore, by employing time-dependent gating, a physician may be presented with an accurate real-time representation of areas of the surface that have already been contacted by the medical device 16′, and corresponding surface map 94, regardless of the phase of the cyclic activity.

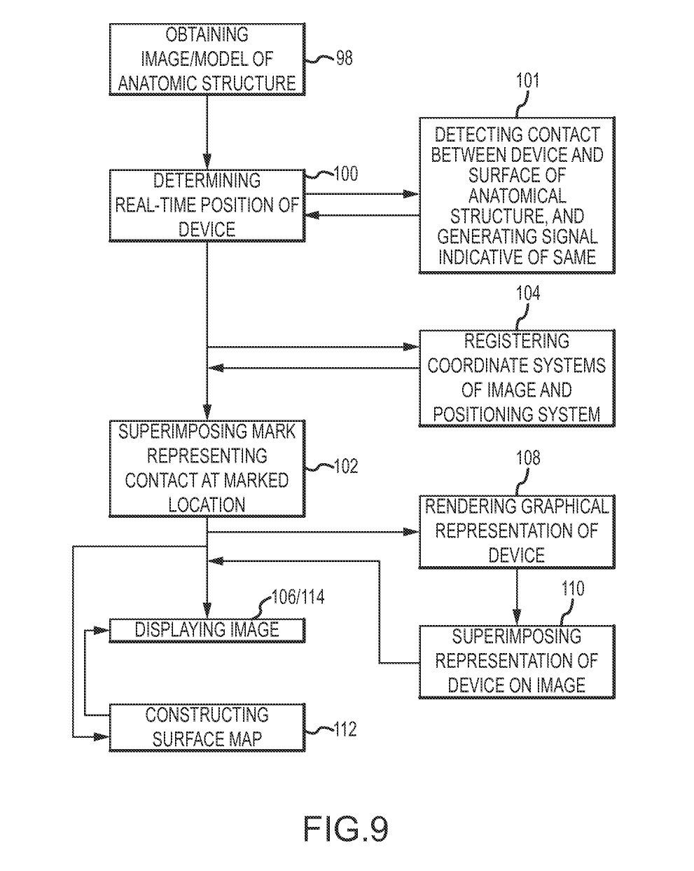

In accordance with another aspect of the invention, and with reference to FIG. 9, a method for mapping a surface of an anatomical structure in real-time is provided. In an exemplary embodiment, the method includes a first step 98 of obtaining an image or model 84 of the anatomical structure, the surface of which is to be mapped. In one exemplary embodiment, step 98 comprises generating a real-time two- or three-dimensional image or model of the anatomical structure, using, for example and without limitation, the methodology described above and illustrated in FIG. 5. In another exemplary embodiment, however, step 98 comprises obtaining a previously acquired image of the anatomical structure.

A second step 100 comprises determining a real-time position of a medical device 16′ when the medical device 16′ contacts the surface of the anatomical structure. In an exemplary embodiment, step 100 comprises determining a real-time P&O of the medical device 16′. The second step 100 includes a substep 101 of detecting contact between the medical device 16′ and the surface of the anatomical structure, and generating a signal indicative of the same.

In a third step 102, a mark 92 corresponding to the position of the medical device 16′ when contact with the surface of the anatomical structure occurred is superimposed onto the image 84. The mark 92 serves to indicate that the surface of the anatomical structure was contacted at the point the mark 92 is disposed. In an exemplary embodiment, step 100 is performed by a positioning system, and therefore, in a fourth step 104, the coordinate system of the image 84 and that of the positioning system are registered with each other.

In an exemplary embodiment, the method further comprises a fifth step 106 of controlling a display device 20 to cause the image 84, and the mark 92 disposed thereon, to be displayed on the display device 20.

The method may further comprise a sixth step 108 of rendering a graphical representation of the medical device 16′; and a seventh step 110 of superimposing the graphical representation of the medical device 16′ onto the image 84.

In an exemplary embodiment, the method may further include an eighth step 112 of constructing, in real-time, a surface map 94 of the anatomical structure based on a plurality of marks 92 superimposed on the image 84. In a ninth step 114, the display device 20 is controlled to cause the constructed surface map 94 to be displayed on the display device 20.

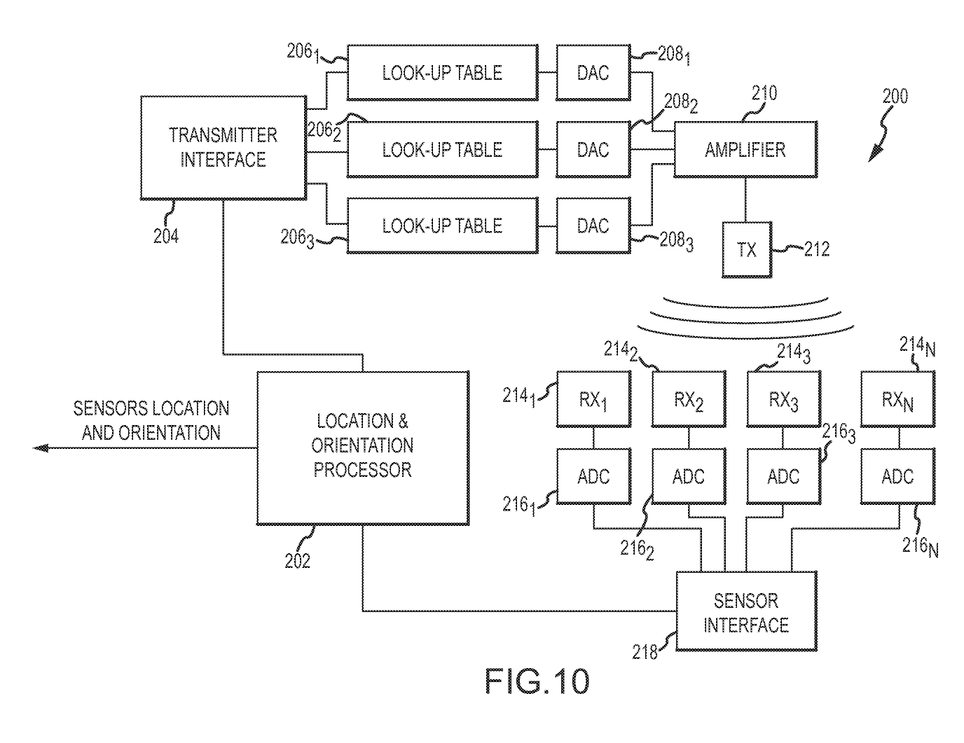

While the description above describes the composition of a MPS (or system 10 comprising an MPS), and the process for determining the P&O of a positioning sensor generally, FIG. 10 is a schematic and block diagram of one specific implementation of a magnetic field-based MPS, designated system 100. Reference is also made to U.S. Pat. No. 7,386,339 entitled “Medical Imaging and Navigation System,” incorporated herein by reference above and portions of which are reproduced below, and which generally describes, at least in part, the gMPS™ medical positioning system commercially offered by MediGuide Ltd. It should be understood that variations in the MPS described below are possible, for example, as also seen by reference to U.S. Pat. No. 6,233,476 entitled “Medical Positioning System,” which was incorporated herein by reference above. Another exemplary magnetic field-based MPS is the Carto™ system commercially available from Biosense Webster, and as generally shown and described in, for example, U.S. Pat. No. 6,498,944 entitled “Intrabody Measurement,” and U.S. Pat. No. 6,788,967 entitled “Medical Diagnosis, Treatment and Imaging Systems,” both of which were incorporated herein by reference above. Accordingly, the following description is exemplary only and not meant to be limiting in nature.

The MPS system 200 includes a location and orientation processor 202, a transmitter interface 204, a plurality of look-up table units 206₁, 206₂ and 206₃, a plurality of digital to analog converters (DAC) 208₁, 208₂ and 208₃, an amplifier 210, a transmitter 212, a plurality of MPS sensors 214₁, 214₂, 214₃ and 214_{N}, a plurality of analog to digital converters (ADC) 216₁, 216₂, 216₃ and 216_{N} and a sensor interface 218.

Transmitter interface 204 is connected to location and orientation processor 202 and to look-up table units 206₁, 206₂ and 206₃. DAC units 208₁, 208₂ and 208₃ are connected to a respective one of look-up table units 206₁, 206₂ and 206₃ and to amplifier 210. Amplifier 210 is further connected to transmitter 212. Transmitter 212 is also marked TX. MPS sensors 214₁, 214₂, 214₃ and 214_{N} are further marked RX₁, RX₂, RX₃ and RX_{N}, respectively. Analog-to-digital converters (ADC) 216₁, 216₂, 216₃ and 216_{N} are respectively connected to sensors 214₁, 214₂, 214₃ and 214_{N} and to sensor interface 218. Sensor interface 218 is further connected to location and orientation processor 202.

Each of look-up table units 206₁, 206₂ and 206₃ produces a cyclic sequence of numbers and provides it to the respective DAC unit 208₁, 208₂ and 208₃, which in turn translates it to a respective analog signal. Each of the analog signals is respective of a different spatial axis. In the present example, look-up table 206₁ and DAC unit 208₁ produce a signal for the X axis, look-up table 206₂ and DAC unit 208₂ produce a signal for the Y axis and look-up table 206₃ and DAC unit 208₃ produce a signal for the Z axis.

DAC units 208₁, 208₂ and 208₃ provide their respective analog signals to amplifier 210, which amplifies and provides the amplified signals to transmitter 212. Transmitter 212 provides a multiple axis electromagnetic field, which can be detected by MPS sensors 214₁, 214₂, 214₃ and 214_{N}. Each of MPS sensors 214₁, 214₂, 214₃ and 214_{N} detects an electromagnetic field, produces a respective electrical analog signal and provides it to the respective ADC unit 216₁, 216₂, 216₃ and 216_{N} connected thereto. Each of the ADC units 216₁, 216₂, 216₃ and 216_{N} digitizes the analog signal fed thereto, converts it to a sequence of numbers and provides it to sensor interface 218, which in turn provides it to location and orientation processor 202. Location and orientation processor 202 analyzes the received sequences of numbers, thereby determining the location and orientation of each of the MPS sensors 214₁, 214₂, 214₃ and 214_{N}. Location and orientation processor 202 further determines distortion events and updates look-up tables 206₁, 206₂ and 206₃, accordingly.

It should be understood that the system 10, particularly the processor 18, as described above may include conventional processing apparatus known in the art, capable of executing pre-programmed instructions stored in an associated memory, all performing in accordance with the functionality described herein. It is contemplated that the methods described herein, including without limitation the method steps of embodiments of the invention, will be programmed in a preferred embodiment, with the resulting software being stored in an associated memory and where so described, may also constitute the means for performing such methods. Implementation of the invention, in software, in view of the foregoing enabling description, would require no more than routine application of programming skills by one of ordinary skill in the art. Such a system may further be of the type having both ROM, RAM, a combination of non-volatile and volatile (modifiable) memory so that the software can be stored and yet allow storage and processing of dynamically produced data and/or signals.

Although only certain embodiments of this invention have been described above with a certain degree of particularity, those skilled in the art could make numerous alterations to the disclosed embodiments without departing from the scope of this disclosure. Joinder references (e.g., attached, coupled, connected, and the like) are to be construed broadly and may include intermediate members between a connection of elements and relative movement between elements. As such, joinder references do not necessarily infer that two elements are directly connected/coupled and in fixed relation to each other. Additionally, the terms electrically connected and in communication are meant to be construed broadly to encompass both wired and wireless connections and communications. It is intended that all matter contained in the above description or shown in the accompanying drawings shall be interpreted as illustrative only and not limiting. Changes in detail or structure may be made without departing from the invention as defined in the appended claims.

What is claimed is:

1. A method for three-dimensionally mapping a volume within a region of interest (ROI) located within a body, comprising the steps of:
tracking the position of an invasive medical device within said ROI in real-time, said tracking step comprising the substeps of:
computing a contour for said medical device as a function of at least one of a shape constraint and a positional constraint; and
translating said contour into a plurality of three-dimensional positions wherein said plurality of three-dimensional positions include both known and virtual positions based on said at least one of said shape and positional constraints;
determining a real-time spatial volume based on at least one of said virtual three-dimensional positions; and
rendering a real-time three-dimensional graphical representation of said spatial volume.

2. The method of claim 1 wherein said plurality of three-dimensional positions corresponding to said contour comprise a first set of three-dimensional positions, said method further comprising:
computing a subsequent contour for said medical device as a function of at least one of a shape constraint and a positional constraint;
translating said subsequent contour into a second set of a plurality of three-dimensional positions wherein said second set of said plurality of three-dimensional positions include both known and virtual positions; and
updating said determined real-time spatial volume if at least one of said plurality of three-dimensional positions within said second set of three-dimensional positions falls outside of said determined real-time spatial volume, said updating comprising revising said real-time spatial volume to include said three-dimensional positions falling outside of the previously determined real-time spatial volume.

3. The method of claim 1 further comprising the step of superimposing said contour of said medical device on said three-dimensional graphical representation of said spatial volume.

4. The method of claim 1 further comprising the steps of:
tracking the motion of said ROI over time; and
compensating for said motion of said ROI in said translation of said contour into said three-dimensional positions.

5. The method of claim 1, further comprising the steps of:
monitoring a cyclic body activity occurring within said ROI;
generating a timing signal based on said monitored cyclic body activity;
tagging each three-dimensional position with a respective time-point in said timing signal; and
wherein said determining step includes determining a respective spatial volume for one or more time-points in said timing signal, and said rendering step includes rendering a three-dimensional graphical representation for each respective spatial volume corresponding to said one or more time-points in said timing signal.

6. A system for three-dimensionally mapping a volume within a region of interest (ROI) of a body, comprising:
a processor, said processor configured to:
compute a contour for a medical device disposed within said ROI as a function of at least one of a shape constraint and a positional constraint;
translate said contour into a plurality of three-dimensional positions wherein said plurality of three-dimensional positions include both known and virtual positions;
determine a real-time spatial volume based on at least one of said virtual three-dimensional positions; and
render a real-time three-dimensional graphical representation of said spatial volume.

7. The system of claim 6, further comprising a medical device having a positioning sensor associated therewith.

8. The system of claim 6 wherein said processor is further configured to superimpose said contour onto said graphical representation of said spatial volume.

9. The system of claim 6 wherein said processor is further configured to monitor the motion of said ROI, and to compensate for said motion of said ROI in said translation of said contour into said three-dimensional positions.

10. The system of claim 6 wherein said plurality of three-dimensional positions corresponding to said contour comprise a first set of three-dimensional positions, said processor is further configured to:
compute a subsequent contour for said medical device as a function of at least one of a shape constraint and a positional constraint;
translate said subsequent contour into a second set of a plurality of three-dimensional positions wherein said second set of said plurality of three-dimensional positions include both known and virtual positions; and
update said determined real-time spatial volume if at least one of said plurality of three-dimensional positions within said second set of three-dimensional positions falls outside of said determined real-time spatial volume, said updating comprising revising said real-time spatial volume to include said three-dimensional positions falling outside of the previously determined real-time spatial volume.

11. The system of claim 6 wherein said processor is further configured to:
monitor a cyclic body activity occurring within said ROI;
generate a timing signal based on said monitored cyclic body activity;
tag each three-dimensional position with a respective time-point in said timing signal; and
generate a respective spatial volume for one or more time-points in said timing signal, and to render a three-dimensional graphical representation for each respective spatial volume corresponding to said one or more time-points in said timing signal.

12. A method for mapping a surface of an anatomical structure in real-time, comprising the steps of:
obtaining an image of said anatomical structure;
determining a real-time position of a medical device when said medical device contacts said anatomical structure; and
superimposing on said image a mark corresponding to said position of said medical device to indicate said anatomical structure was contacted at the point on said anatomical structure where said mark is disposed.

13. The method of claim 11, wherein said obtaining step comprises one of:
generating one of an image and a model of said anatomical structure; and
obtaining one of a previously acquired image of said anatomical structure and a previously acquired model of said anatomical structure.

14. The method of claim 11, wherein said determining step is performed by a positioning system, and said method further comprises the step of registering the coordinate system of said image with the coordinate system of said positioning system.

15. The method of claim 11 further comprising the steps of:
rendering a graphical representation of said medical device; and
superimposing said graphical representation of said medical device onto said displayed image.

16. The method of claim 11 further comprising the step of constructing, in real-time, a surface model of said anatomical structure based on a plurality of said marks superimposed on said image or model.

17. A system for mapping a surface of an anatomical structure, comprising:
a processor configured to:
obtain an image of said anatomical structure;
receive a signal indicative of a medical device contacting said surface of said anatomical structure;
determine a real-time position of said medical device responsive to said signal when said signal is indicative of said medical device contacting said anatomical structure, and
superimpose onto said image of said anatomical structure a mark corresponding to said position of said medical device to indicate said anatomical structure was contacted by said medical device at the point on said anatomical structure where said mark is disposed.

18. The system of claim 17, further comprising a sensing element associated with said medical device configured to generate a signal indicative of contact between said medical device and said anatomical structure.

19. The system of claim 17 further comprising an imaging system configured to generate said image of said anatomical structure, and to communicate said image to said processor.

20. The system of claim 17, wherein said processor is configured to generate said image of said anatomical structure.

21. The system of claim 17, wherein said processor is further configured to construct, in real-time, a surface model of said anatomical structure based on a plurality of said marks superimposed on said image.
